(19) United States
(12) Patent Application Publication
(10) Pub. No.: US 2024/0050896 A1
(43) Pub. Date: Feb. 15, 2024

(54) COATED CROSSFLOW FILTER MEMBRANES AND FILTRATION SYSTEMS FOR FRACTIONAL SEPARATION OF BIOLOGICAL MATERIAL

(71) Applicants: AXBIO INC., Santa Clara, CA (US); SHENZEN XBIOME BIOTECH CO. LTD., Shenzhen (CN)

(72) Inventors: IGOR IVANOV, SANTA CLARA, CA (US); HUI TIAN, SANTA CLARA, CA (US); YAN TAN, SHENZHEN (CN); TING GAO, SHENZHEN (CN); CONG ZHANG, SHENZHEN (CN)

(21) Appl. No.: 18/233,185

(22) Filed: Aug. 11, 2023

Related U.S. Application Data
(60) Provisional application No. 63/397,562, filed on Aug. 12, 2022.

(51) Int. Cl. B01D 61/18; B01D 63/08

(52) U.S. Cl. CPC B01D 61/18; B01D 63/082; B01D 2311/263; B01D 2313/08; B01D 2313/54; B01D 2319/06

(57) ABSTRACT

Coated crossflow filter membranes, crossflow filters comprising the coated filter membranes and crossflow filtration systems for separating material such as biological material sensitive to temperature, dissolved gases, and mechanical damage are disclosed.

This application claims the benefit under 35 U.S.C. § 119(e) of U.S. Provisional Application No. 63/397,562 filed on Aug. 12, 2022, which is incorporated by reference in its entirety.

### FIELD

The invention relates to coated crossflow filter membranes, crossflow filters comprising the coated crossflow filter membranes and crossflow filtration systems for separating biological material sensitive to temperature, dissolved gases, and mechanical damage.

### BACKGROUND

It can be desirable to separate biological materials such as cells, viruses, bacteria, fungal spores, and proteins from a suspension. This can be accomplished using crossflow filtration methods. The viability of the separated biological material can be compromised during the separation process by exposure to temperature, dissolved gases, and mechanical damage.

### SUMMARY

According to the present invention, crossflow filter membranes comprise a porous substrate and a coating overlying the porous substrate.

According to the present invention, fractional filtration systems comprise a sample preparation module; a first filtration module, wherein the first filtration module is fluidly coupled to the sample preparation module; a second filtration module, wherein the second filtration module is fluidly coupled to the first filtration module; and a target material processing module, wherein the target material processing module is fluidly coupled to the second filtration module; wherein each of the first filtration module and the second filtration module independently comprises a crossflow filter.

According to the present invention, methods of separating a target biological material from a suspension comprise using the fractional filtration system according to the present invention.

### DETAILED DESCRIPTION OF THE DRAWINGS

The drawings described herein are for illustration purposes only. The drawings are not intended to limit the scope of the present disclosure.

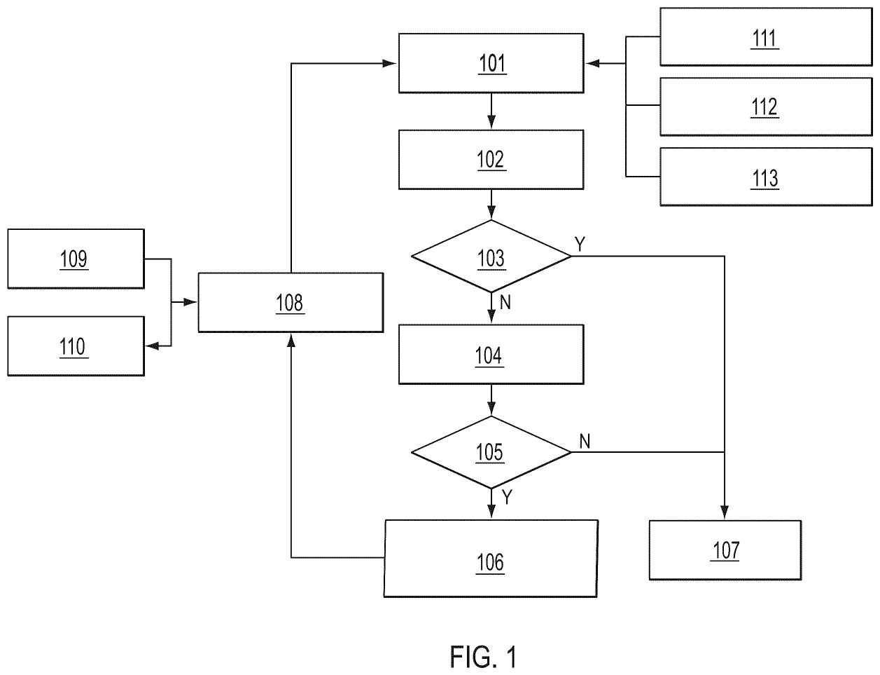

FIG. 1 shows a schematic diagram of an example of a crossflow filtration system provided by the present disclosure.

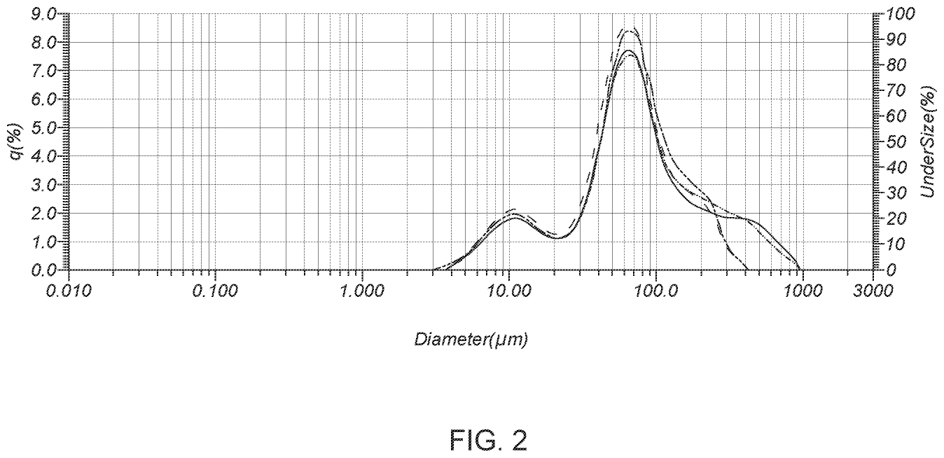

FIG. 2 shows particle size distributions for the 20 wt % soybean paste feed.

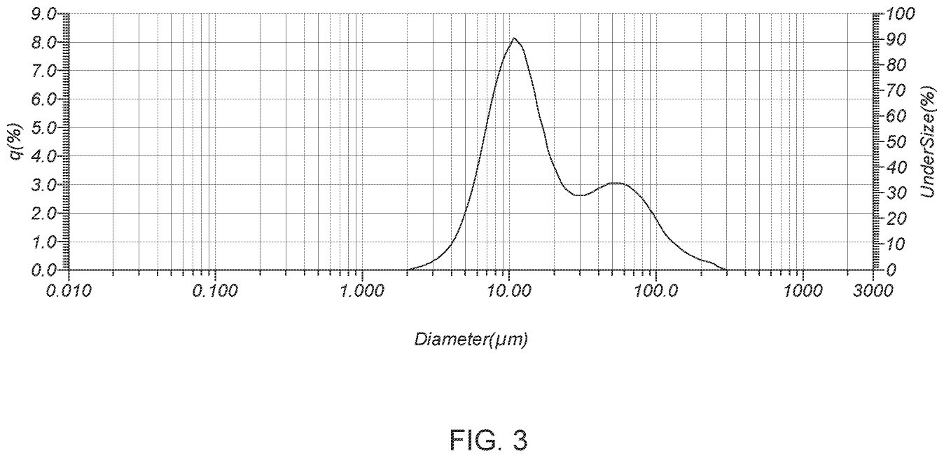

FIG. 3 shows a particle size distribution of the filtrate obtained using a 50 MG crossflow filter.

### DETAILED DESCRIPTION

A crossflow filtration system provided by the present disclosure can be configured to separate viable biological material from a medium. A crossflow filtration system can also be referred to as a tangential flow filtration system. A viable biological material can be capable of reproducing. For example, a crossflow filtration system provided by the present disclosure can be used to separate viable cells or sub-populations of cells, viruses, bacteria, fungal spores, and/or proteins from a medium.

During separation from a suspension, the viability of a biological material can be compromised, for example, by exposure to certain temperatures, dissolved oxygen, and mechanical stress. To minimize damage to target biological material, and to maintain the viability of the biological material during and after separation from the suspension, a crossflow filtration system provided by the present disclosure can provide a controlled thermal environment, minimize dissolved oxygen concentrations, and can minimize mechanical damage potentially caused by passage of the target biological material thorough pores of a filter membrane.

A crossflow filtration system can be configured to maintain the integrity of a target biological material such as a population of cells. For example, it can be desirable to separate a population of cells from a biological sample without compromising the cell walls. Following separation, the integrity of the target biological material can be maintained, and the target biological material may or may not be viable.

A fractional crossflow filtration system provided by the present disclosure can be configured to concentrate a target material having dimensions within predetermined dimension limits. For example, a sample such as a suspension containing the target material can be filtered to remove material having dimensions greater than a defined average size limit and material having dimensions less than a defined average size limit, thereby concentrating a target material within a range of the dimensions between the upper and lower size limits.

An example of a fractional biological separation system provided by the present disclosure is shown in FIG. 1. The separation system shown in FIG. 1 includes a loading/unloading module 108, a sample preparation module 101, a first filtration module 102, a first process element 103, a second filtration module 104, a second process module 105, a target biological material processing module 106, and a waste module 107.

Each of the modules is fluidly coupled to other modules and flow of a suspension through the system is controlled by a series of values and pumps, which are not shown in FIG. 1.

Each of the process modules 103 and 105 represent independent controls for routing a crossflow filter retentate or filtride (large solids) or a crossflow filter permeate or filtrate (small solids) to either the waste module 107, to a subsequent (second) filtration module 104, or to target material processing module 106. For example, a first process module 103 can route a first filtration module 102 retentate containing large solids to waste module 107 and route a first filtration module 102 permeate to a second filtration module 104. Second process module 105 can be configured to route a second filtration module 104 permeate to waste module 107 and route a second filtration module 104 retentate to target material processing module 106.

A biological sample material container 109 and a target material collection container 110 can be fluidly coupled to loading/unloading module 108.

A temperature control media module 111, a gas control module 112, and a service media module 113 can be fluidly coupled to sample preparation module 101 as shown in FIG. 1. The temperature control media module 111, a gas control module 112, and a service media module 113 can be fluidly coupled to one or more other modules of the system including modules 102-110.

A sample preparation module can include a mixing chamber. The mixing chamber can include inlets for addition of a material sample 101 and for adding a solvent such as water. The mixing chamber can be thermally controlled. The mixing chamber can include an agitator configured to suspend solids in a solvent and to disperse agglomerates. The solvent can be a biological media or buffer.

The mixing chamber can include one or more apparatus for suspending a target biological material in a solution. The mixing chamber can include, for example, a homogenizer, an agitator, an inert gas purge, and apparatus for delivering additional agents and/or materials to the mixing chamber to form a suspension comprising the biological material sample.

A process solvent reservoir can be coupled to the mixing chamber to mix the biological material with a suitable solvent such as, for example, water, buffer, solvent (hydrophilic or hydrophobic), cryoprotectant, cryoprotectant, and/or bacteria growth media.

The temperature of the mixing chamber and the contents can be thermally controlled, for example from 95° F. to 100° F. (35° C. to 38° C.).

The environment of the mixing chamber environment and of the biological material suspension can be configured to maintain the viability of the target biological material.

The outlet of the sample preparation module can be fluidly coupled to the first filtration module through a first fluid pump.

A fractional filtration system provided by the present disclosure can include two or more filtration modules.

Each filtration module can independently comprise a crossflow filter.

A filtration module can further comprise a mixing chamber configured to maintain the suspension of the biological material. The mixing chamber can precede and be fluidly coupled to the crossflow filter.

A filtration module includes an inlet coupled to a preceding processing module such as the sample preparation module or to a previous filtration module.

A filtration module can be temperature controlled.

A filtration module can be configured to maintain a desired concentration of dissolved gases. For example, a filtration chamber can be coupled to a gas source such as a N₂ or inert gas source at a pressure, for example, from 40 psig to 80 psig (275 kpa to 551 kpa), such as 50 psig to 70 psig (345 kpa to 483 kpa).

A filtration module can comprise an outlet fluidly coupled to a subsequent filtration module, to the waste module, and/or to a target material processing module.

A filtration system provided by the present disclosure can include more than two filtration modules.

A first filtration module can separate a suspension into a first permeate and a first retentate, where the permeate refers to the portion of the suspension that passes through the filter membrane, and the first retentate refers to the portion of the suspension that does not pass through the crossflow filter. The first permeate comprises material that passes through the pores of the filter membrane and has a smaller average size that the material contained in the first retentate.

The first permeate can then be directed into a second crossflow filter and separated into a second permeate and a second retentate, where the material in the second permeate has smaller dimensions that that of the second retentate. Either the second permeate or the second retentate can contain the target biological material.

The filtered suspension containing the target biological material can then be routed into another crossflow filter and either small-dimensioned or large-dimensioned material relative to the target biological material can be separated from the suspension containing the target biological material.

The suspension containing the target biological material can be routed to one or more additional filtration modules to further separate and/or concentrate the target biological material.

A suspension containing the target biological material can be re-routed through one or more previous filtration modules to repeat one or more of the filtrations and further concentrate and/or refine the size range of the target biological material.

A suspension that does not contain the target biological material can be routed through one or more previous filtration modules to extract target biological material that was not previously filtered.

Suspensions that do not contain the target biological material can be routed to a waste module.

A suspension or suspensions containing the target biological material can be routed to a target material processing module.

A target material processing module can be configured to wash, further concentrate, and/or resuspend the target biological material.

For example, the target biological material can be washed with biological buffer solutions, concentrate by centrifugation, and resuspend the target biological material in a biological medium. This process can be repeated as appropriate.

The target material processing module can be coupled to a solvent source and a gas source.

The target material processing module can be temperature controlled.

A filtration module provided by the present disclosure can comprise a crossflow filter.

A crossflow filter can comprise a filter membrane.

A crossflow filter membrane can comprise any suitable porous substrate such as a metal, metal alloy, ceramic, thermoplastic, or thermoset. A crossflow filter can comprise a metal, metal alloy, ceramic, glass, polymer, or polymer composite porous substrate.

A crossflow filter membrane can have an average pore size selected to separate a certain target biological material from a suspension. A filtration system provided by the present disclosure can comprise two or more crossflow filters with each of the crossflow filters selected to separate or pass a selected portion of the suspension and configured to separate and/or concentrate a target biological material.

A coated crossflow filter membrane can have an average pore size, for example, from 0.01 μm to 1,000 μm, from 0.1 μm to 1,000 μm, from 1 μm to 1,000 μm, from 1 μm to 500 μm, from 1 μm to 250 μm, from 5 μm to 200 μm, from 10 μm to 150 μm, or from 10 μm to 100 μm, from 10 μm to 50 μm, from 1 μm to 10 μm, from 0.1 μm to 10 μm, from 0.1 μm to 5 μm, from 0.1 μm to 1 μm, or from 0.1 μm to 0.5 μm.

A coated crossflow filter membrane can have an average pore size, for example, greater than 0.01 μm, greater than 0.05 μm, greater than 0.1 μm, greater than 0.5 μm, greater than 1 μm, greater than 5 μm, greater than 10 μm, greater than 50 μm, greater than 100 μm, greater than 250 μm, or greater than 500 μm.

A coated crossflow filter membrane can have an average pore size, for example, less than 1,000 μm, less than 500 μm, less than 100 μm, less than 50 μm, less than 10 μm, less than 5 μm, less than 1 μm, less than 0.5 μm, less than 0.1 μm, or less than 0.05 μm.

Surfaces of a crossflow filter membrane provided by the present disclosure can comprise a coating. The coating can be any suitable coating that is configured to minimize or prevent mechanical damage caused, for example, by shear stress as a target biological material passes through the pores of a crossflow filter membrane. A coating can be a conformal coating, meaning that the coating conforms to the contoured surfaces of the crossflow filter membrane to form a thin, pinhole free layer, and provides a coating layer having a substantially homogeneous thickness. A conformal coating can cover and effectively smooth the surfaces of the membrane pores and thereby minimize damage to the target biological material caused by impact against the surfaces of the filter.

A filter membrane coating can have a thickness, for example, from 0.01 μm to 250 μm, from 0.01 μm to 200 μm, from 0.01 μm to 150 μm, from 0.01 μm to 100 μm, from 0.01 μm to 10 μm, or from 0.01 μm to 1 μm.

A filter membrane coating can have an average thickness, for example, less than 250 μm, less than 200 μm, less than 150 μm, less than 100 μm, less than 50 μm, less than 10 μm, less than 5 μm, less than 1 μm, less than 0.5 μm, or less than 0.1 μm.

A filter membrane coating can have an average thickness, for example, greater than 0.05 μm, greater than 0.1 μm, greater than 0.5 μm, greater than 1 μm, greater than 5 μm, greater than 10 μm, greater than 50 μm, greater than 100 μm, greater than 150 μm, or greater than 200 μm.

A filter membrane coating can be selected to have biochemical, chemical and/or physical properties that facilitate the ability of a target biological material to pass through the pores of a filter membrane without being mechanically damaged. For example, a filter membrane coating can be selected to have a desired hydrophilicity, elasticity, zeta potential, chemical activity, biochemical activity, toxicity and other properties required for separation, extraction or separation of a specific material.

The properties of a filter membrane coating can be selected to enhance flow of the target biological material through the filter membrane and to minimize the shear forces on the target biological material as the material passes through the pores of the filter membrane.

The properties of a filter membrane coating can be selected based, for example, on the pH of an aqueous a solution, the hydrophilicity of the solution,

A filter membrane coating can have a hydrophilic surface, wherein the hydrophilicity of the surface is determined by measuring the contact angle according to ASTM F22-21. For example, a hydrophilic coating surface can have a contact angle less than 90°, less than 80°, less than 70°, less than 60°, less than 50°, less than 40°, or less than 30°, determined according to ASTM F22-21. For example, a hydrophilic coating surface can have a contact angle from 30° to 90°, from 30° to 80°, from 30° to 70°, from 30° to 60°, or from 30° to 50°, determined according to ASTM F22-21.

A filter membrane coating can have a hydrophobic surface wherein the contact angle is greater than 90°, determined according to ASTM F22-21. For example, a hydrophobic surface can have a contact angle greater than 90°, greater than 100°, greater than 110°, or greater than 120°, determined according to ASTM F22-21. A hydrophobic surface can have a contact angle, for example, from 90° to 130°, from 90° to 120°, from 90° to 110°, or from 90° to 100°, determined according to ASTM F22-21. A filter membrane coating can have an oleophilic surface or an oleophobic surface.

A hydrophilic coating surface can have a critical surface tension greater than 45 dynes/cm and a hydrophobic surface can have a critical surface tension less than 35 dynes/cm. An oleophobic surface can have critic surface tension less than 20 dynes/cm.

A membrane coating can comprise, for example, a silicone-based (polysiloxane-based) coating. A silicone coating can be functionalized, for example, to enhance adhesion to a filter membrane, to render the exterior surface of the coating hydrophilic, to render the exterior surface of the coating hydrophobic, or to provide a coating surface having a desired zeta potential. The surface of the silicone coating can be configured to facilitate flow of the target biological material through coated pores, to minimize adhesion of material to the surfaces of the coated pores, and/or to facilitate the ability to clean and sterilize the coated filter membrane.

A silicone coating can be covalently bonded to the porous substrate.

A metal, ceramic, carbon, or polymer filter membrane can be exposed to a plasma such as an oxygen plasma to enhance adhesion of the coating to the filter membrane.

To enhance adhesion to the filter membrane a prepolymer such as a silicone prepolymer used to form the coating can comprise pendent polyalkoxysilane groups.

A coating can be applied to the surfaces of the filter membrane using, for example, atomic layer deposition (ALD) or chemical vapor deposition (CVD).

A crossflow filtration module provided by the present disclosure can be configured to provide an electrical gradient across the filter membrane.

A fractional filtration system provided by the present disclosure can comprise ancillary components such as valves, check valves, pumps such as progressive cavity pumps, Y strainers, a steam trap, level switches, fluid pressure sensors, gas pressure sensors, temperature sensors, flow sensors, agitators, and sprayers. These and other ancillary components can be situated at suitable locations throughout the system to monitor and control processing of the biological material sample to separate and/or concentrate the target biological material.

A filtration system provided by the present disclosure can include a processor configured to control the filtration process. The processor can be electrically interconnected or operatively connected to the various sensors such as temperature sensors, pressure sensors, level sensors, and flow sensors and to the control elements such as the values used to route the sample material, filtration retentates, and filtration permeates, through the system, and to control the temperature, pressure, levels, and flow rates of the solutions through the system.

A filtration system provided by the present disclosure can be configured to filter and concentrate a target biological material fraction of a biological material sample. The disclosed filtration system can be used to concentrate a target biological material fraction of a material sample that can be degraded outside of a certain temperature range, by dissolved gases in the sample, and/or by mechanical stress.

For example, biological samples such as biological cells and bacteria can be damaged during the filtration process such as in the high shear stress environment during passage through the filter membrane.

A target material can be a viable biological material such as, for example, a population of cells, a population of bacteria, a population of virus, a population of fungi, population of spores, or a population of organelle.

A target biological material can have an average dimension, for example, from 0.1 μm to 100 μm, from 1 μm to 75 μm, from 5 μm to 50 μm, from 10 μm to 50 μm, from 0.1 μm to 1.0 μm, from 0.1 μm to 0.5 μm, from 1 μm to 20 μm, or from 1 μm to 10 μm. A biological material can have an average dimension, for example, less than 100 μm, less than 80 μm, less than 60 μm, less than 40 μm, less than 20 μm, less than 10 μm, less 5 μm, or less than 1 μm.

To facilitate the ability of the fractional cross-filtration system provided by the present disclosure to maintain the integrity of the target material, the system is sealed from the environment.

A system provided by the present disclosure can further provide a temperature control module to control the temperature at each of the modules and conduits between modules.

A system provided by the present disclosure can comprise a gas control module configured to control the concentration of dissolved gases in the material sample. The gas control module can be configured to control the level of gases in the material sample to prevent oxidation. The system can comprise one or more gas sources such as oxygen O₂, nitrogen N₂, and/or an inert gas such as Ar coupled to one or more of the material sample processing modules. The gas control module can control the introduction of gas into the material sample based on input from gas sensors. The concentration of gases within the system can be selected to maintain the integrity and/or viability of the biological samples.

A system provided by the present disclosure can comprise a service functionality module. A service functionality module can be configured to control operations such as cleaning one or more of the modules and fluid conduits between processing steps. A service functionality module can be configured to sterilize the system between processing steps.

A fractional biological separation system provided by the present disclosure can be implemented to process any suitable volume of sample material. For example, the system can be implemented on a production scale, pilot-plant scale, laboratory scale, or be implemented using microfluidics.

The system can be implemented to process sample material volumes of less than 1,000 liters, less than 500 liters, less than 100 liters less than 50 liters, less than 1 liter, less than 500 mL, less than 100 mL, less than 50 mL, less than 10 mL, less than 5 mL, less than 1 mL, less than 0.5 mL, or less than 0.1 mL.

A system provided by the present disclosure can be used to separate a target biological material from a suspension of a complex biological material.

For example, a system provided by the present disclosure can be used to separate target bacteria from a fecal sample.

Bacteria associated with the gut biome are known to affect the efficacy of therapy and produce therapeutically effective compounds. Separating target bacteria populations derived from the gut biome such as from fecal samples can help in understanding the role of the bacteria in homeostasis and disease and in developing more effective therapies.

### ASPECTS

The invention is further defined by one or more of the following aspects.

Aspect 1. A crossflow filter membrane comprising a substrate and a coating overlying the substrate.

Aspect 2. The crossflow filter membrane of aspect 1, wherein the substrate comprises a porous meta, a porous ceramic, a porous glass, a porous polymer, a porous composite, or a combination of any of the foregoing.

Aspect 3. The crossflow filter membrane of any one of aspects 1 to 2, wherein the substrate has an average pore diameter from 1 μm to 200 μm.

Aspect 4. The crossflow filter membrane of any one of aspects 1 to 3, wherein the substrate comprises an average pore diameter from 0.01 μm to 1,000 μm.

Aspect 5. The crossflow filter membrane of any one of aspects 1 to 4, wherein the coating comprises a conformal coating.

Aspect 6. The crossflow filter membrane of any one of aspects 1 to 5, wherein the coating is covalently bonded to the substrate.

Aspect 7. The crossflow filter membrane of any one of aspects 1 to 6, wherein the coating is elastomeric.

Aspect 8. The crossflow filter membrane of any one of aspects 1 to 7, wherein the coating has a shear modulus greater than 0.3 MNm⁻².

Aspect 9. The crossflow filter membrane of any one of aspects 1 to 8, wherein the coating comprises a silicone.

Aspect 10. The crossflow filter membrane of any one of aspects 1 to 9, wherein the coating comprises a functionalized coating.

Aspect 11. The crossflow filter membrane of aspect 10, wherein the functionalized coating is hydrophilic.

Aspect 12. The crossflow filter membrane of aspect 10, wherein the functionalized coating is hydrophobic.

Aspect 13. The crossflow filter membrane of aspect 10, wherein the functionalized coating is oleophobic.

Aspect 14. The crossflow filter membrane of aspect 10, wherein the functionalized coating has a zeta potential from −100 mV to +100 mV.

Aspect 15. The crossflow filter membrane of any one of aspects 1 to 14, wherein the coating is configured to reduce shear stress of a biological material passing through the crossflow filter membrane.

Aspect 16. The crossflow filter membrane of any one of aspects 1 to 15, wherein the coating has an average thickness from 0.1 μm to 250 μm.

Aspect 17. A crossflow filter comprising the coated crossflow filter membrane of any one of aspects 1 to 16.

Aspect 18. A fractional filtration system comprising: a sample preparation module; a first filtration module, wherein the first filtration module is fluidly coupled to the sample preparation module; a second filtration module, wherein the second filtration module is fluidly coupled to the first filtration module; and a target material processing module, wherein the target material processing module is fluidly coupled to the second filtration module; wherein each of the first filtration module and the second filtration module independently comprises a crossflow filter.

Aspect 19. The fractional filtration system of aspect 18, wherein each of the first filtration module and the second filtration module independently comprises the crossflow filter of aspect 17.

Aspect 20. The fractional filtration system of any one of aspects 18 to 19, wherein the first filtration module is configured to remove material having an average dimension greater than an upper dimension from a suspension of biological material.

Aspect 21. The fractional filtration system of any one of aspects 18 to 20, wherein the second filtration module is configured to remove material having an average dimension less than a lower dimension from a suspension of biological material.

Aspect 22. The fractional filtration system of any one of aspects 18 to 21, comprising one or more additional filtration modules, wherein each of the one or more additional filtration modules is fluidly coupled to the first filtration module or the second filtration module.

Aspect 23. The fractional filtration system of aspect 22, wherein each of the one or more additional filtration modules comprises the crossflow filter of aspect 17.

Aspect 24. The fractional filtration system of any one of aspects 18 to 23, comprising a loading/unloading module fluidly coupled to the sample preparation module.

Aspect 25. The fractional filtration system of aspect 24, wherein the fractional filtration system comprises a target material processing module, wherein the target material processing module is fluidly coupled to the second filtration module and to the loading/unloading module.

Aspect 26. The fractional filtration system of any one of aspects 18 to 25, comprising a temperature control module, wherein the temperature control module is operatively coupled to one or more of the modules.

Aspect 27. The fractional filtration system of aspect 26, wherein the temperature control module comprises a temperature control media.

Aspect 28. The fractional filtration system of any one of aspects 18 to 27, comprising: a gas control module; and a service media control module, wherein each of the gas control module and the service media control module is independently fluidly coupled to one or more of the modules.

Aspect 29. The fractional filtration system of aspect 28, wherein the gas control module is configured to control the partial pressure of N₂, Ar, CO₂, H₂, or a combination of any of the foregoing.

Aspect 30. The fractional filtration system of any one of aspects 28 to 29, wherein the service media control module is configured to control the flow of cleaning solvent, sterilization solvent, or a combination thereof.

Aspect 31. The fractional filtration system of any one of aspects 18 to 30, wherein each of the first filtration module and the second filtration module is independently fluidly coupled to a waste module.

Aspect 32. The system of any one of aspects 18 to 31, comprising a source material container fluidly coupled to the loading/unloading module.

Aspect 33. The fractional filtration system of aspect 32, comprising a target material container fluidly coupled to the loading/unloading module.

Aspect 34. The fractional filtration system of any one of aspects 18 to 33, wherein the fractional filtration system is sealed from the ambient environment.

Aspect 35. The fractional filtration system of any one of aspects 18 to 34, wherein the fractional filtration system is configured to separate a target biological material from a suspension.

Aspect 36. The fractional filtration system of aspect 35, wherein the target biological material comprises a population of cells, a population of bacteria, a population of virus, population of spores, or a population of organelle.

Aspect 37. The fractional filtration system of any one of aspects 35 to 36, wherein the target biological material is viable.

Aspect 38. The fractional filtration system of any one of aspects 35 to 36, wherein the target biological material is capable of reproducing.

Aspect 39. The fractional filtration system of any one of aspects 18 to 38, wherein the fractional filtration system is configured to separate a biological material having a target dimension.

Aspect 40. The fractional filtration system of aspect 39, wherein the target dimension is within a range from 1 μm to 100 μm.

Aspect 41. The fractional filtration system of any one of aspects 18 to 40, comprising a processor operatively coupled to the system.

Aspect 42. The fractional filtration system of aspect 41, wherein, the system comprises control devices values, pumps, level sensors, level controls, flow sensors, flow controls, temperature sensors, temperature controls, pressure sensors, pressure controls, or a combination of any of the foregoing; and the processor is operatively coupled to the control devices.

Aspect 43. A method of separating a target biological material from a suspension comprising using the fractional filtration system of any one of aspects 18 to 42.

Aspect 44. The method of aspect 43, wherein the target biological material is selected from a population of cells, a population of bacteria, a population of virus, a population of spores, a population of organelle, or a combination of any of the foregoing.

Aspect 45. The method of any one of aspects 43 to 44, wherein the separated target biological material is viable.

Aspect 46. The method of any one of aspects 43 to 44, wherein the separated biological material is capable of reproducing.

Aspect 47. The method of any one of aspects 43 to 46, wherein the suspension comprises a sample biological material.

Aspect 48. The method of aspect 47, wherein the sample of biological material comprises a gastrointestinal material, gut biome, stool, blood, saliva, tissue, or combination of any of the foregoing.

Aspect 49. The method of aspect 47, wherein the sample of biological material comprises a soil sample, a water sample, an environmental sample, or a plant sample.

### EXAMPLES

Embodiments provided by the present disclosure are further illustrated by reference to the following examples, which describe filtration systems and methods of using the filtration systems. It will be apparent to those skilled in the art that many modifications, both to materials, and methods, may be practiced without departing from the scope of the disclosure.

### Example 1

### Processing of a Biological Material Using a Multistage Crossflow Filtration System

A multistage crossflow filtration system was evaluated using a soybean paste slurry as an example of a biomass sample. All testing occurred at ambient temperature (20° C. to 25° C.).

As the feed to the crossflow filtration system, a 20 wt % soybean paste slurry in distilled water was prepared.

In Test 1, using a MG 50 porous metal crossflow filter, the measured filtrate particle size ranged from 2.6 μm to 262 μm. Filtrate flow data was not collected due to improper flow transducer setup; however, a maximum transmembrane pressure (TMP) of 4.2 psi (29.0 kpa) was reached. The feed flow rate was measured to be approximately 3.5 gpm (13.25 lpm) throughout the test. The filtrate from Test 1 was then used as a feed for Tests 2 and 3.

In Test 2 a MG 0.2 crossflow filter was used to remove filtride within a particle size range from 0.2 μm to 50 μm. The resulting filtride contained particulates ranging in size from 2.6 μm to 229 μm, while the filtrate slurry contained particulates ranging in size from 0.07 μm to 10.1 μm. A TMP of 16 psi (110.3 kpa) was required to generate filtrate flow through the porous media. At this pressure, significant extrusion of the soft soybean paste solids occurred, leading to the high upper particle sizes within the range. The average filtrate flow was 0.012 gpm (0.045 L/min) with a flux of 0.05 gpm/ft² (2 lpm/m²).

The filtrate from Test 1 was used as the feed for Test 3 in which a MG 0.5 MG crossflow filter was used. Testing resulted in filtride particulates ranging in size from 3 μm to 262 μm. The filtrate particulates ranged in size from 0.2 μm to 77 μm. At a TMP of 10 psi, the filtrate flow was 0.008 gpm (0.030 L/min) with a flux of 0.04 gpm/ft² (1.6 lpm/m²). As for Tests 1 and 2, the relatively high TMP resulted in high particulate breakthrough.

Overall, the capability of the multistage crossflow filtration system to remove particulates less than 0.2 μm and greater than 50 μm was demonstrated. By flowing the filtrate through a 50 MG filter and a 0.5 MG filter at the lowest TMPs that allowed filtrate flow, the filtrate had a particle size ranging from 0.2 μm to 77 μm.

The soft nature of the solid component of the soybean slurry resulted in significant breakthrough, especially at higher TMPs. In all testing, extremely low filtrate flow rates resulted from the high viscosity and compressibility of the process slurry. Filtration of such soft materials is difficult for any crossflow filter or direct barrier technology. However, with a low TMP the compressibility of the bacterial solids in a biomass sample can be minimized. Also, use of a soft coating material on the filter can also reduce breakthrough.

Multistage crossflow filtration studies were performed on a representative sample of soybean paste using a 0.2 MG, 0.5 MG, and 50 MG crossflow filter cartridges. The RnD system uses a single 0.5 inch OD×24 inch (1.3 cm×61 cm) long porous element. The ID of the cartridge is ⅜-inch (9.52 mm), yielding a filtration area of 0.2 ft² (0.0186 m²).

A schematic of the test set-up is shown in FIG. 1. The solids of soybean paste were mixed in deionized (DI) water to a 20 wt % concentration. The filtrate, or liquid that passed through the porous metal filter 50 MG crossflow filter cartridge was used for further testing on the 0.2 MG and 0.5 MG crossflow filter cartridges. The particle size distribution of the filtride, or material left that did not pass through the 0.2 MG porous filter, was measured using a Horiba Particle Size Distribution analyzer. Particle size analysis was undertaken for the feed used in Test 1, the filtrate in Test 1, and the filtride and filtrate for Test 2.

A RnD Crossflow System available from Swagelock with 24-inch (61 cm) 0.5 MG, 0.2 MG, and 50 MG porous metal crossflow filter cartridges was used in the tests.

A Horiba Particle Size Distribution analyzer was used to measure the particle size of the solid samples.

The procedure used to separate a solid slurry is summarized as follows.
1. Twenty-four (24) L of feed containing 20 wt % solids of soybean paste in deionized water was prepared.
2. The particle size range of the feed slurry was measured.
3. A 50 MG porous crossflow filter was installed, and clean flow data was measured to establish base line system conditions.
4. In Test 1-1 the feed was filtered using a 50 MG crossflow filter while measuring the pressure drop of the mainstream feed line and the transmembrane pressure (TPM) across the wall of the crossflow filter. Six (6) L of filtrate was collected.
5. In Test 1-2 the feed was filtered using a 50 MG crossflow filter while measuring the pressure drop of the mainstream feed line and the TPM across the wall of the crossflow filter. Six (6) L of filtrate was collected and combined with the filtrated from Test 1-1 to provide a total of 12 L of filtered feed for Test-2 and Test-3.
6. The particle size range of the 50 MG filtrate was measured using a Horiba Particle Size Analyzer.
7. For Test-2, a 0.2 MG crossflow filter was installed, and clean flow data were measured to establish base line system conditions
8. The feed from Test 1-1 and Test 1-2 were filtered using the 0.2 MG crossflow filter while measuring the pressure drop of the mainstream feed line and the TPM across the wall of the crossflow filter.
9. A particle size distribution analysis was performed on the 0.2 MG filtrate and on the filtride.
10. A 0.5 MG porous element was installed in the crossflow filter system and the clean flow data was determined to establish base line system conditions
11. In Test 3 the filtrate from Test 1-1 and Test 1-2 was used as the feed to the 0.5 MG crossflow filter while measuring the pressure drop of the mainstream feed line and the TPM across the wall of the crossflow filter.
12. The particle size distribution of the 0.5 MG filtrate and the feed remaining in the tank after testing was measured.

Tests 1-1 and 1-2: 50 MG Filtration.

Tests 1-1 and 1-2 were conducted using a 50 MG crossflow filter with 24 L of feed. The particle size range of the feed was from 4.47 μm to 1000 μm. The filtrate of the 50 MG test had a particle size ranging from 2.59 μm. To 262.37 μm. With a 50 MG porous crossflow filter, the size of the filter pores was up to from 55 μm to 60 μm. The presence of solids well beyond the theoretical maximum pore size of the 50 MG crossflow filter results from the presence of soft solids. When the transmembrane pressure (TMP) is increased, these soft solids deform. This resulted in extrusion of the deformed soft solids through the pores of the crossflow filter and/or plugging of pores. The particles sizes of the feed for the 50 MG crossflow filter and of the filtrate are shown in Table 1.

A total of 12 L of filtrate was collected from the 50 MG crossflow filter to be used as the main feed in Test 2 and Test 3.

Test 2: 0.2 MG Filtration.

Test 2 was conducted using 0.2 MG crossflow filter. The filtride, or recirculated slurry remaining in the feed tank after filtrate is removed, is the concentrated slurry. The particle size of the filtride solids was from 2.59 μm to 229.07 μm. Consistent with the maximum pore size for a 0.2 MG crossflow filter of approximately 1.5 μm, the solids size exclusion range meets expectations.

The particle size range of the filtrate is characterized by solids ranging in size from 0.07 μm to 10.1 μm. Similar to the results obtained using the 50 MG crossflow filter, the particle size range of the filtrate solids indicates breakthrough.

Test 3: 0.5 MG Filtration.

Test 3 was conducted using 0.5 MG crossflow filter. The filtride particle size ranged from 2.97 μm to 262.37 μm. The 0.5 MG filtrate particle size ranged from 0.22 μm 77.3 μm. During Test 3, the TMP was limited to 10 psi (68.9 kpa) to decrease the risk of deforming solids and extruding solids through or plugging the porous media. Similar to the particle size results obtained for Test 1 and 2, breakthrough is evident in the filtrate when using 0.5 MG.

During testing all three media grades were recovered after backwashing the solids into the recirculation tank. The coarser solids passed through the 0.2 MG and 0.5 MG crossflow filters. This was caused by having to increase the TPM to 10 psi (68.9 kpa) to achieve flow on the filtrate line. As the solids were deformable and soft, it appears that the soybean paste was extruded at high transmembrane pressures.

The filter test conditions are summarized in Table 1.

The measured particle size ranges for the feed, filtride and filtrate for the tests is summarized in Table 2.

TABLE 1
Filtration test conditions.

| Test | 1-1 | 1-2 | 2 | | 3 | |
|---|---|---|---|---|---|---|
| Filter Media Grade (MG) | 50 | 50 | 0.2 | | 0.5 | |
| Cycle Time (min) | 40 | 28 | 283 | | 131 | |
| Mainstream Flow (min) | 3.24-3.70 | 3.05-3.19 | 4.49-5.62 | | 2.80-3.26 | |
| Mainstream Flow (gpm) (lpm) | ¹ — | ¹ — | 0.012 | (0.045) | 0.008 | (0.030) |
| Avg. Filtrate Flow (gpm) (lpm) | ¹ — | ¹ — | 0.0545 | (0.204) | 0.0364 | (0.136) |
| Transmembrane Pressure (psi) (kpa) | 0.3-4.2 (2.06- | 3.6-4.1 (24.8- | 1.0-16.2 (6.9- | | 1.7-10.9 (11.7- | |
| | 29.0) | 28.3) | 111.7) | | 75.1) | |
| Backwash Pressure (psi) (kpa) | 45 (310) | 45 (310) | 45 | (310) | 45 | (310) |

| | | | | |
|---|---|---|---|---|
| Mainstream Flow (L/min) | 12.3-14.0 | 11.5-12.1 | 17.0-21.3 | 10.6-12.3 |
| Avg. Filtrate flow (L/min) | ¹ — | ¹ — | 0.045 | 0.03 |
| Avg. Flux (L/min/m²) | ¹ — | ¹ — | 2.15 | 1.51 |
| Transmembrane Pressure (kg/cm) | 0.021-0.3 | 0.25-0.29 | 0.07-1.14 | 0.12-0.77 |
| Backwash Pressure (kg/cm²) | 3.16 | 3.16 | 3.16 | 3.16 |

¹ Not measured.

TABLE 2
Measured feed, filtride and filtrate particle size ranges.

| Test | 1-1 | 1-2 | 2 | 3 |
|---|---|---|---|---|
| Filter Media Grade (MG) | 50 | 50 | 0.2 | 0.5 |
| Feed Solids Range (μm) | 4.5-1,000 | 4.5-1,000 | 2.6-262.4 | 2.6-262.4 |
| Filtride Solids Range | | | 2.6-229.1 | 3.0-262.4 |
| (μm) | | | | |

| | | | |
|---|---|---|---|
| Filtrate Solids Range | 2.6-262.4 | 0.1-10.1 | 0.2-77.3 |
| (μm) | | | |

The particle size distribution for the 20 wt % soybean paste feed is shown in FIG. 2 and corresponds to a particle size range from 4.5 μm to 1,000 μm.

The particle size distribution for the filtrate following separation using a 50 MG crossflow filter is shown in FIG. 3 and corresponds to a particle size range from 2.6 μm to 262 μm.

Finally, it should be noted that there are alternative ways of implementing the embodiments disclosed herein. Accordingly, the present embodiments are to be considered as illustrative and not restrictive, and the claims are not to be limited to the details given herein but may be modified within the scope and equivalents thereof.

What is claimed is:

1. A crossflow filter membrane comprising a porous substrate and a coating overlying the porous substrate.

2. The crossflow filter membrane of claim 1, wherein the coating is elastomeric.

3. The crossflow filter membrane of claim 1, wherein the coating has a shear modulus greater than 0.3 MNm⁻².

4. The crossflow filter membrane of claim 1, wherein the coating comprises a silicone.

5. The crossflow filter membrane of claim 1, wherein the coating comprises a functionalized coating.

6. A crossflow filter comprising the coated crossflow filter membrane of claim 1.

7. A fractional filtration system comprising:
a sample preparation module;
a first filtration module, wherein the first filtration module is fluidly coupled to the sample preparation module;
a second filtration module, wherein the second filtration module is fluidly coupled to the first filtration module; and
a target material processing module, wherein the target material processing module is fluidly coupled to the second filtration module;
wherein each of the first filtration module and the second filtration module independently comprises a crossflow filter.

8. The fractional filtration system of claim 7, wherein each of the first filtration module and the second filtration module independently a crossflow filter membrane comprising a porous substrate and a coating overlying the porous substrate.

9. The fractional filtration system of claim 7, wherein the first filtration module is configured to remove material having an average dimension greater than an upper dimension from a suspension of biological material.

10. The fractional filtration system of claim 7, wherein the second filtration module is configured to remove material having an average dimension less than a lower dimension from a suspension of biological material.

11. The fractional filtration system of claim 7, comprising a loading/unloading module fluidly coupled to the sample preparation module.

12. The fractional filtration system of claim 7, comprising a temperature control module, wherein the temperature control module is operatively coupled to one or more of the modules.

13. The fractional filtration system of claim 7, comprising:
a gas control module; and
a service media control module,
wherein each of the gas control module and the service media control module is independently fluidly coupled to one or more of the modules.

14. The fractional filtration system of claim 7, wherein the fractional filtration system is sealed from the ambient environment.

15. The fractional filtration system of claim 7, wherein the fractional filtration system is configured to separate a target biological material from a suspension.

16. The fractional filtration system of claim 15, wherein the target biological material is viable.

17. The fractional filtration system of claim 15, wherein the target biological material is capable of reproducing.

18. A method of separating a target biological material from a suspension comprising using the fractional filtration system of claim 7.

19. The method of claim 18, wherein the target biological material is selected from a population of cells, a population of bacteria, a population of virus, a population of spores, a population of organelle, or a combination of any of the foregoing.

20. The method of claim 18, wherein separated target biological material is viable.
